(19) United States
(12) Patent Application Publication
(10) Pub. No.: US 2025/0128059 A1
(43) Pub. Date: Apr. 24, 2025

(54) DETERMINING RESTLESS LEGS SYNDROME SYMPTOMS BASED ON SENSED LIMB MOVEMENT

(71) Applicants: Stephanie Kayla Rigot, Pleasanton, CA (US); Jonathan David Charlesworth, San Francisco, CA (US)

(72) Inventors: Stephanie Kayla Rigot, Pleasanton, CA (US); Jonathan David Charlesworth, San Francisco, CA (US)

(21) Appl. No.: 18/922,200

(22) Filed: Oct. 21, 2024

Related U.S. Application Data
(60) Provisional application No. 63/544,845, filed on Oct. 19, 2023.
(60) Provisional application No. 63/663,608, filed on Jun. 24, 2024.
(60) Provisional application No. 63/663,650, filed on Jun. 24, 2024.

(51) Int. Cl. A61N 1/36; A61N 1/04

(52) U.S. Cl. CPC A61N 1/36031; A61N 1/0456; A61N 1/36021; A61N 1/36034

(57) ABSTRACT

An approach to detecting patient limb movement during electrostimulation can include delivering an electrostimulation waveform, e.g., generated by an electrostimulation electronics unit, having a frequency within a range of 500 Hz to 10,000 kHz, and having an average root-mean squared current exceeding 5 milliamps. Concurrently, motion data can be received from an inertial measurement unit (IMU) during the therapy session. Based on this motion data, a timing, frequency, or amplitude of limb movements, exhibited by the patient over the therapy session, can be estimated such as to provide insight into patient response and potential symptoms of a sleep disorder.

### CLAIM OF PRIORITY

This application claims priority to U.S. Provisional Application Ser. No. 63/544,845, filed on Oct. 19, 2023, U.S. Provisional Application Ser. No. 63/663,608, filed on Jun. 24, 2024, and U.S. Provisional Application Ser. No. 63/663,650, filed on Jun. 24, 2024, which are each incorporated by reference herein in its entirety, and the benefit of priority of each of which is claimed herein.

### STATEMENT REGARDING FEDERALLY SPONSORED RESEARCH OR DEVELOPMENT

This invention was made with government support under R44 NS117294 awarded by the National Institute of Neurological Disorders and Stroke division of the National Institutes of Health. The government has certain rights in the invention.

### BACKGROUND

Electrical nerve stimulation can be used to treat one or more conditions, such as chronic or acute pain, epilepsy, depression, bladder disorders, or inflammatory disorders. Certain neurological disorders can be attributed to overactivity of sensory or other peripheral nerve fibers which can disrupt quality of life, and/or the processing of such neural activity in the brain.

Restless Legs Syndrome (RLS) and Periodic Limb/Leg Movement Disorder (PLMD) are two such neurological conditions that can significantly affect sleep in human subjects. RLS (which can also be called Willis-Ekbom Disease (WED)) subjects can experience uncomfortable tingling sensations in their lower limbs (legs) and, less frequently in the upper limbs (arms). RLS is characterized by an uncontrollable urge to move the affected limb(s). Such sensations can often be temporarily relieved by moving the limb voluntarily but doing so can interfere with the RLS subject's ability to fall asleep. PLMD subjects can experience spontaneous movements of the lower legs during periods of sleep, which can cause the PLMD subject to wake up. RLS can be a debilitating sleep disorder and can be comorbid with other sleep disorders such as insomnia or sleep apnea syndrome (SAS).

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 depicts an example of a limb wearable electrostimulation device for treating at least one of RLS or PLMD.

FIG. 2A depicts an example of a limb wearable neurostimulation device, arranged on a patient leg concurrently with an electromyography (EMG) electrode or concurrently with an inertial measurement unit (IMU).

FIG. 2B is a flowchart showing signal processing which can be performed to process or analyze received motion data.

FIG. 3A is a chart showing smoothed motion data, including a change in movement parameter over time.

FIG. 3B is a chart showing unsmoothed motion data, including a change in movement parameter over time.

FIG. 3C is a chart showing a change in movement parameter over time, including an adaptive noise floor.

FIG. 4 is a chart showing a threshold-based technique for determining an indication of an individual limb movement.

FIG. 5A is a chart showing electromyography (EMG) movement parameters plotted over time.

FIG. 5B is a chart showing accelerometer movement parameters plotted over time.

FIG. 5C is a chart showing gyroscopic movement parameters plotted over time.

FIG. 6 is a chart showing several approaches to estimating a patient symptom of Restless Legs Syndrome during concurrent delivery of an electrostimulation waveform to the patient.

FIG. 7 is a chart showing a PLM frequency linear regression approach to predicting numerical rating scale (NRS) ratings of subjective RLS symptom severity.

FIG. 8 is a flowchart describing a process for detecting patient limb movement during a transcutaneous therapy session that includes delivery of neurostimulation therapy on a human patient.

FIG. 9 illustrates generally an example of a block diagram of a machine.

### DETAILED DESCRIPTION

In healthcare, Restless Legs Syndrome (RLS) management can involve diagnosis, changes related to medication, changes to nonpharmacological treatments (e.g., adjusting electrostimulation parameters), or long-term tracking to identify if a patient is developing a tolerance to a particular therapy solution. Reliably monitoring of RLS symptom frequency and severity can be challenging, especially via approaches that do not track or observe any kind of objective biomarker of RLS and instead rely on subjective patient feedback and self-diagnosis.

RLS can, however, be associated with certain quantifiable, patient movements such as stereotyped involuntary periodic leg movements during sleep (PLMS) periodic leg movements during wake (PLMW), voluntary leg movements during wake (e.g., limb movements performed by a patient to alleviate RLS symptoms), and motion data indicating postural changes during sleep. For example, PLMS are repetitive movements that occur in series throughout the night following specific temporal patterns. Such PLMS have been shown to be related to RLS severity and may be a viable method to objectively monitor RLS symptoms and response to treatment. Certain approaches to specifically detecting PLMS can involve in-clinic polysomnography, which can involve a challenge of being resource-intensive, e.g., to make it unfeasible in certain situations for monitoring RLS.

Approaches to treating Restless Legs Syndrome (RLS) and Periodic Limb Movement Disorder (PLMD) can include pharmacological interventions and non-pharmacological interventions such as lifestyle changes. Such approaches can involve challenges of efficacy, side effects, tolerance to therapy, or challenges associated with patient non-compliance. Neurostimulation devices can be useful in certain medical applications to treat neurological and physiological conditions. Such devices typically employ electrical stimulation to modulate neural activity in specific regions of the body. Thus, neurostimulation can provide a treatment modality for certain limb-related disorders, such as Restless Legs Syndrome (RLS) and Periodic Limb Movement Disorder (PLMD), such as for alternative or additional treatment to pharmacological agents, lifestyle changes, massage therapy, compression therapy, etc. Wearable neurostimulation devices can include or otherwise be worn with an inertial measurement unit (IMU, e.g., including an accelerometer and a gyroscope) or other instrument for detecting movement at or near a patient limb. The present inventors have recognized the benefits of configuring such a wearable device such that it can (e.g., concurrently) provide RLS therapy and provide an objective estimate of RLS symptoms, thus performing multiple functions, such as a diagnostic, therapy, and therapy management tool. This document describes a technique for detecting patient limb movement during a transcutaneous neurostimulation therapy session, including delivery of high-frequency electrostimulation provided concurrently with motion data analysis to estimate a timing and frequency of limb movements. Such limb movements can be processed and analyzed for identifying symptoms of conditions such as restless legs syndrome (RLS) or periodic limb movement disorder (PLMD).

For example, the technique can involve a wearable electrostimulation device for generating and delivering an electrostimulation waveform, via electrodes, to an external target body site of the patient during a therapy session. Herein, “therapy session” can include time directly before and after electrostimulation (e.g., within minutes of commencing or ending electrostimulation) and time between intermittent stimulations that are delivered during the session, during which therapy session the wearable electrostimulation device is worn on a patient leg, and is not strictly limited to an electrostimulation period during which active electrostimulations are always occurring. This waveform can be generated via an electrostimulation electronics unit of the wearable electrostimulation device, such as including a frequency within the range of 500 Hz to 10,000 kHz and an average root-mean squared current greater than 5 mAᵣₘₛ. These parameters can be selected to provide effective neurostimulation while inhibiting patient discomfort such as paresthesia at or near an electrode-skin interface.

During the therapy session, including but not limited to time periods that are concurrent with the electrostimulation, the technique can involve receiving motion data from an inertial measurement unit (IMU). The IMU, e.g., including one or more accelerometers or gyroscopes, can capture detailed information about the patient's limb movements during the therapy session. This collection of motion data can help facilitate a comprehensive analysis of patient response to the treatment. For example, motion data can be received and processed, such as to estimate of the timing, frequency, or amplitude of limb movements exhibited by the patient over the therapy session. This estimation process can involve algorithms that can identify specific types of movements, such as periodic limb movements (PLMs) voluntary movements made by the patient to relieve RLS symptoms, or voluntary movements made by the patient that indicate a state of arousal or awakening.

In an example, the technique can involve establishing or adjusting at least one electrostimulation waveform parameter based on the estimated timing and frequency of limb movements. Such an adaptive approach can help facilitate a personalized treatment protocol that can be fine-tuned in response to the patient's specific movement patterns.

For example, the technique can involve processing of the motion data for movement detection and classification. For instance, the motion data can be identified or classified over a period, e.g., according to a specified threshold, such as to determine the start and end of individual limb movements. For example, a “starting” (e.g., marking a beginning of a sensed, individual limb movement) threshold can be defined by motion data meeting one or more criteria, such as by exceeding a specified accelerometer threshold value, a specified gyroscope threshold value, or both. Also, an “ending” (e.g., marking an end or decay of a sensed, individual limb movement) threshold can be established when the motion data falls beneath specified accelerometer and gyroscope values.

To enhance the accuracy of movement detection, the technique can include signal processing steps such as filtering or smoothing the motion data, summing respective vectors from multiple sensor axes to calculate a vector magnitude, or identifying candidate limb movements based on vector magnitude. In an example, the technique can include evaluating certain candidate limb movements against a reference data set of validated limb movements, such as to help distinguish genuine limb movements from artifacts. In an example, the technique can include one or more adaptive techniques to account for varying conditions during the therapy session. For example, the technique can establish an adaptive noise floor that adjusts according to a specified moving window throughout the therapy period. This aspect can help facilitate accurate movement detection even in the presence of changing background noise or patient activity levels.

By integrating high-frequency electrostimulation with motion analysis, exemplary techniques described herein can provide a comprehensive approach to monitoring and treating conditions characterized by detected limb movements. For example, such an approach can be used to help direct attention from customer support or healthcare providers specifically toward patients who are not showing an expected or desired relief from RLS symptoms during or following electrostimulation. Such an approach can facilitate development of personalized and effective treatment protocols in the management of RLS, PLMD, or other sleep-related disorders.

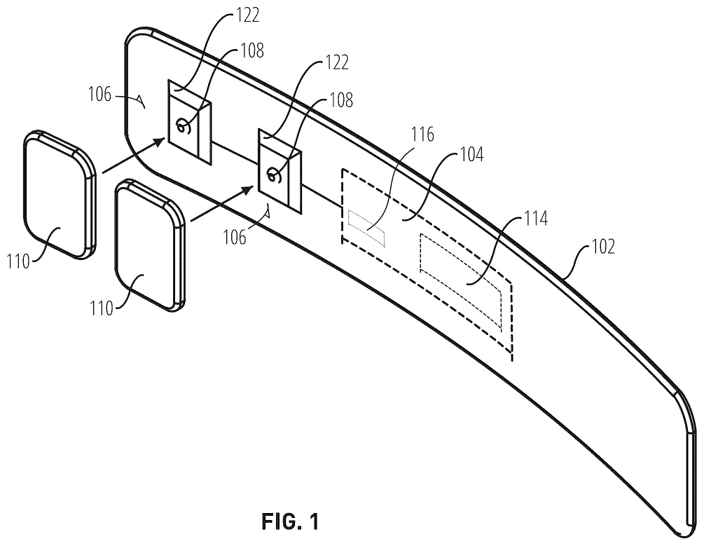

FIG. 1 depicts an example of a limb wearable electrostimulation device for treating at least one of RLS or PLMD. One approach to neurostimulation involves Transcutaneous Electrical Nerve Stimulation (TENS). TENS devices generate low-voltage electrical currents that stimulate nerves in the area of pain via delivery of the current to an external body location such as patient skin. TENS devices involve electrical pulses delivered via a capacitive discharge, such as at relatively frequencies ranging from 1 Hertz (Hz) to 250 Hz and maximum current amplitudes below 50 mA. A challenge with neurostimulation via TENS is that TENS is generally delivered at considerably low duty cycles, e.g., 5-10%, between long relaxation periods. Such low duty cycles can involve relatively low root means squared (RMS) current actually delivered to the patient, e.g. 1-3 mA, and therefore, relatively low charge delivery. As such, TENS devices can deliver relatively low energy, but generally remain insufficient to generate action potentials above, e.g., neural firing thresholds. Thus, while the electrical current delivered by TENS delivers insufficient energy for a therapeutic stimulation motor nerves (e.g., activating proprioceptive afferents), it can serve as a kind of counter-irritant that masks pain signals.

This document describes devices and methods involving a high frequency stimulation (HFS) approach, such as neurostimulation involving the use of frequencies greater than 500 Hz or greater than 1000 Hz. Such “high” frequencies are above the typically perceived pain threshold and generates little or no paresthesia. Unlike TENS, where the frequency is usually constant and the duty cycle is modulated (e.g., 5-10%), HFS employs a modulated duty cycle or burst mode at a much higher current amplitude to directly stimulate peripheral nerves. However, HFS can avoid the “sizzle and burn” effects encountered in many TENS applications. Specifically, due to the higher frequency of HFS, the charge density can be reduced while delivering the same charge or energy, as compared to TENS, thus the heat dissipated at the electrode interface is reduced in HFS. Producing HFS in a wearable, battery-powered device presents a considerable challenge in terms of power management, heat dissipation, and overall device efficiency. The present inventors have conceived of techniques for HFS neurostimulation in a limb wearable neurostimulation device to treat at least one of RLS or PLMD, such as to help overcome the above-identified challenges. The devices and methods discussed herein can help facilitate safe, comfortable delivery of a waveform that induces tonic motor activation (TOMAC) of a patient muscle, without inducing uncomfortable paresthesia, without waking a patient from sleep, or without significantly heating (e.g., maintaining less than 10° greater than patient skin temperature) patient skin.

As depicted in FIG. 1, a wearable electrostimulation device 102 can include an electrostimulation electronics unit 104 and one or more electrostimulation electrodes 106. The wearable electrostimulation therapy device 102 can deliver electrostimulation therapy to skin of a subject via charge-dispersing materials such as the electrode pads 110, which help form the electrodes 106. In an example, the electrode pads 110 can be removably couplable to the wearable electrostimulation device 102. The electrode pads 110 can each be attached to a pairing surface 122 of the wearable electrostimulation device 102. The pairing surface 122 can include the electrode terminal 108 e.g., disposed therein, and the electrode terminal 108 can be electrically connected to the electrostimulation electronics unit 104. In an example, two or more electrode pads 110 are respectively paired to corresponding pairing surfaces 122 including a corresponding one of electrode terminals 108. In another example, one electrode pad 110 can be paired to an electrode pairing surface 122 containing more than one electrode terminal 108, or one electrode pad can span multiple electrode pairing surfaces 122 containing one or more electrode terminals 108. In an example, the electrode pad 110 can be removable for, e.g., hygienic maintenance, electrode maintenance such as rehydrating, or disposal.

The wearable electrostimulation device 102 can be worn by the subject and can include or use the electrostimulation electronics unit 104 coupled to the electrodes 106, such as for transcutaneously delivering an electrostimulation signal. The wearable electrostimulation device 102 can be sized and shaped to be able to be attached or held to a body location of the subject, e.g., a leg, arm, foot, waist, neck, head, or chest of the human subject. In an example, the wearable electrostimulation device 102 can include or use a strap to help hold the electrodes 106 to the skin of the subject. While electrodes are generally described herein with a focus on providing electrostimulation to a subject, the electrodes can alternatively or additionally be used such as to help detect or measure one or more biosignals or biopotentials from the subject. A particular electrode 106 can include or use an electrode terminal 108 and an electrode pad 110. For example, the electrode terminal 108 can receive a capacitively-coupled (e.g., coupled using series DC-blocking capacitors, e.g., in a charge-balanced arrangement) electrostimulation signal from the electrostimulation electronics unit 104, and can deliver a resulting electrostimulation signal to the skin of the subject, such as via the electrode pad 110. In an example, multiple electrodes, such as two electrodes 106, can be used. For a bipolar electrode example having two electrodes 106, e.g., in close proximity to each other, this can include a first electrode that can serve as an anode and a second electrode, such as which can serve as a cathode. Also, a plurality of electrodes 106 can be arranged to form a multi-electrode group, matrix, or array such as for one or both of sensing or for delivering the electrostimulation signal to the skin of the subject. In an example, each electrode terminal 108 can include an electrode contact fixed to the wearable electrostimulation device 102 and each corresponding electrode pad 110 can be removably couplable to the device 102 by the end-user. In other examples, the electrode pad can be more permanently fixed to the wearable electrostimulation device 102. Because the current density of the electrostimulation signal at the electrode terminal 108 may be larger than desired, the electrode pad 110 can include an embedded or other arrangement of electrical conductors that can help distribute the electrostimulation signal current over a larger effective surface area for delivery to the subject at the skin-electrode interface.

For example, the patient electrodes can be positioned at or near a targeted nerve (e.g., a sural nerve, a peroneal nerve, a tibial nerve, etc.). The electrode pad can be formed of a hydrogel, a hydrophilic polymer such as polyvinyl alcohol (PVA), carbon, textiles, or other types of conductive or dielectric gels, a polymer, or a textile. In an example, two wearable electrostimulation devices 102 can be worn bilaterally, such as on different limbs of a patient to provide bilateral electrostimulation. The device as a whole can be a wearable solution, such as featuring an elongate strap that can substantially encircle the limb. The strap can hold the electrodes, which are electrically connected to the electrostimulation electronics unit, against the patient's skin. The strap can be sized and shaped and formed of a material for flexibility and comfort, such as including a battery housing and an electronics housing arranged at different locations along the strap length to facilitate articulation of the strap when worn.

An individual wearable electrostimulation device 102 can include an electrostimulation electronics unit 104 communicatively coupled to the electrostimulation electrode(s) 106. The wearable electrostimulation device 102 can be worn by the subject and can include or use the electrostimulation electronics unit 104 coupled to the electrodes, such as for transcutaneously delivering an electrostimulation signal. Herein, the term high frequency electrostimulation (HFS) can refer to certain high-frequency (greater than 500 Hertz, such as greater than 1 kilohertz) electrostimulation waveforms capable of inducing tonic motor activation (TOMAC) when applied at an external target body location of the patient. This is distinct from other approaches to electrostimulation, such as TENS or implantable devices that operate at considerably different parameters that are generally not compatible with sleep or for treating RLS or PLMD, such as parameters that involve sensory perception of the patient and that do not induce TOMAC in a patient muscle. In an example, the HFS devices and methods herein include use of a capacitive coupling to apply HFS that induces TOMAC in a particular muscle without attempting to surround current injected in skin with a large opposite current, such as is the case in TENS paradigms, as evident during electromyogram monitoring. In an example, the electrostimulation electronics unit 104 can include or use processor circuitry 116, e.g., including a microprocessor, microcontroller, programmable logic circuit, or the like, such as can be powered by an onboard battery or other power source. In an example, the onboard battery can be coupled to a power converter circuit, such as can include one or more of a buck power converter circuit, a boost power converter circuit, a buck-boost power converter circuit, or other inductive or capacitive or other circuit for converting the battery voltage and current to a desired output voltage and current such as for delivering electrostimulation to the subject via the patient electrodes. The electrostimulation electronics unit 104 can also include or use an electrostimulation waveform generator circuit, configured to receive a converted power signal from the power converter circuit, and can generate a suitable electrostimulation waveform, such as an HF electrostimulation waveform such as described herein. The electrostimulation waveform generator can also be configured to generate the HF electrostimulation controlled-current waveform including a current amplitude, e.g., controlled by the processor circuitry 116, within a range of 5 milliamperes to 30 milliamperes (e.g., at a level of 5 mA, 10 mA, 15 mA, 20 mA, 25 mA, or 30 mA, or finer resolution if desired). An example of an electrostimulation waveform generator is described in US Patent Publication Serial No. 2019/0083784 which is incorporated by reference herein in its entirety, including for its teaching of an electrostimulation device and techniques for waveform generation to treat at least one symptom of RLS or PLMD.

In an example, the wearable electrostimulation device 102 can include or be communicatively coupled to an inertial measurement unit (IMU) 114. In an example, the IMU 114 can be arranged on or within the wearable electrostimulation device 102, such as within the electrostimulation electronics unit 104 or communicatively coupled to a printed circuit board assembly (PCBA) of the wearable electrostimulation device 102. The IMU 114 can include an accelerometer, a gyroscope, a magnetometer, or a combination thereof. The IMU 114 can transmit linear acceleration data or angular velocity data, such as for a plurality of axis (e.g., an x-axis, a y-axis, and a z-axis). In an example, the IMU 114 can include at least a 6-axis inertial measurement unit. In an example, the wearable electrostimulation device 102 can include processor circuitry 116 including instructions that, when executed by a processor, cause the processor to commence recording, storage, or transmission of motion data from the IMU 114.

The motion data received from the IMU can be used by the processor circuitry 116, such as to determine past or present movement or to help predict future movement of a patient limb, e.g., at or near a patient nerve target. In an example, motion data can be used by the processor circuitry 116 to control at least one parameter of the HF electrostimulation signal. For example, delivery of a HF electrostimulation signal can be triggered or initiated upon receiving motion data indicating a specified number (e.g., greater than about 5 or about 10) of leg movements within a specified first sensing duration (e.g., within a duration that can be specified at 1 minute, 2 minutes, 5 minutes, or the like), is received from the IMU. For example, the leg movements can be periodic leg movements, movements/positional information that is correlative with patient subjective RLS symptoms during wake, or movements/positional information that is correlative with a likelihood of a patient waking. This can be useful, for example, in a PLMD patient who can experience leg twitches or motions while sleeping, such as to prompt the device to automatically turn on (or increase) therapy to help mitigate symptoms to help the patient stay asleep. Also, at least one parameter of the HF electrostimulation signal (e.g., a frequency, amplitude, pulse-width, etc.) can be established or adjusted based on motion data, corresponding with leg twitches or motions, within a specified second sensing duration, such as received via the IMU. For example, the second sensing duration can be concurrent with the therapy session during which HF electrostimulation signal is being delivered to the patient (e.g., persist beyond a duration that can be specified at 5 minutes, 10 minutes, or the like). In an example, electrostimulation can be turned off (or can be ramped down or ramped off) where motion data is received indicating fewer than a specified number (e.g., less than about 10, about 5, or about 2) of leg twitches or motions or motion data not indicative of leg twitches or motions, the motion data received over a third sensing duration (e.g., within a duration that can be specified at 1 minute, 2 minutes, 5 minutes, or the like).

In an example, the electrostimulation electronics unit 104 can include an output port such as USB port or Bluetooth circuitry for transmission of raw or processed motion data from the IMU 114 to other electronic systems, such as for further processing via algorithms or machine learning models and for displaying results to, e.g., a patient or a healthcare provider. Alternatively or additionally, the electrostimulation electronics unit 104 can also include storage and processor circuitry 116 for executing an algorithm for transforming raw motion data from the IMU 114 into estimates of limb movements (e.g., PLMs).

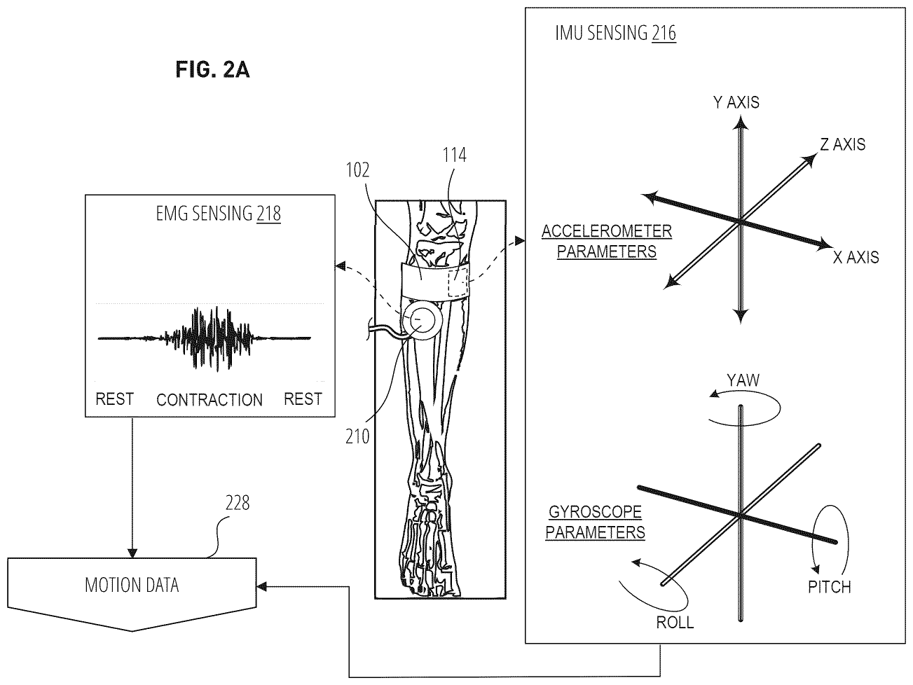

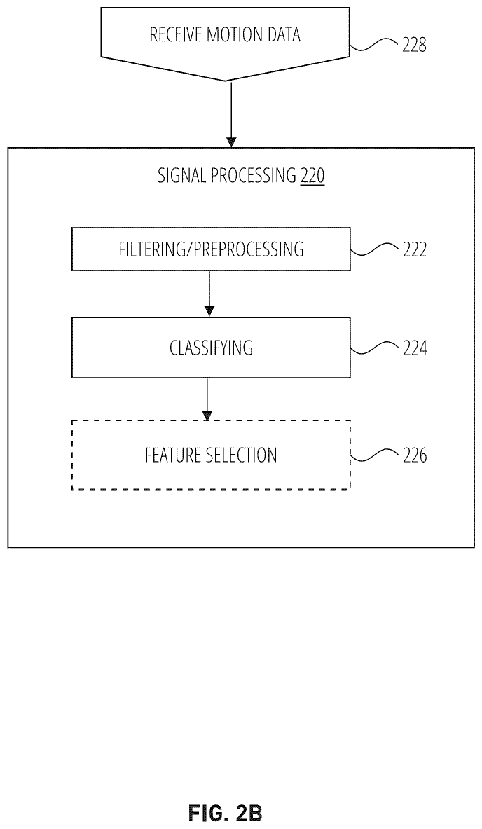

FIG. 2A depicts an example of a limb wearable electrostimulation device, arranged on a patient leg together with an electromyography (EMG) electrode or together with an inertial measurement unit (IMU). The wearable electrostimulation device 102 can include or be worn by the patient concurrently with at least one of an IMU inertial measurement unit (IMU) 114. For example, the IMU 114 can include an accelerometer configured for taking velocity measurements along at least three different axis and can alternatively or additionally include a gyroscope configured to take rotational measurements (e.g., roll, pitch, and yaw) along at least three different axes. The wearable electrostimulation device 102 can also include (alternatively or additionally to the IMU 114) an electromyography (EMG) electrode 210. An example of such an EMG electrode 210 is described in US Patent Publication Serial No. US 2021/0100998, which is incorporated by reference herein in its entirety for its teaching of surface electromyography (sEMG) electrodes for personalized patient feedback related to electrostimulation. As shown in FIG. 2A, at least one of IMU Sensing 216 or EMG Sensing 218 can be controlled by the processor circuitry 116 (as depicted in FIG. 1) such as to record motion data 228.

FIG. 2B is a flowchart showing signal processing which can be performed to process or analyze received motion data (e.g., from an IMU). an example, the IMU sensing/preprocessing 216 can include transforming raw motion data from the IMU 114 into estimates of limb movements, such as transforming raw motion data into estimates of PLMs. In an example, such an IMU Sensing 216 can be performed by the processor circuitry 116 or other componentry, e.g., directly in embedded software on the PCBA of the wearable electrostimulation device 102.

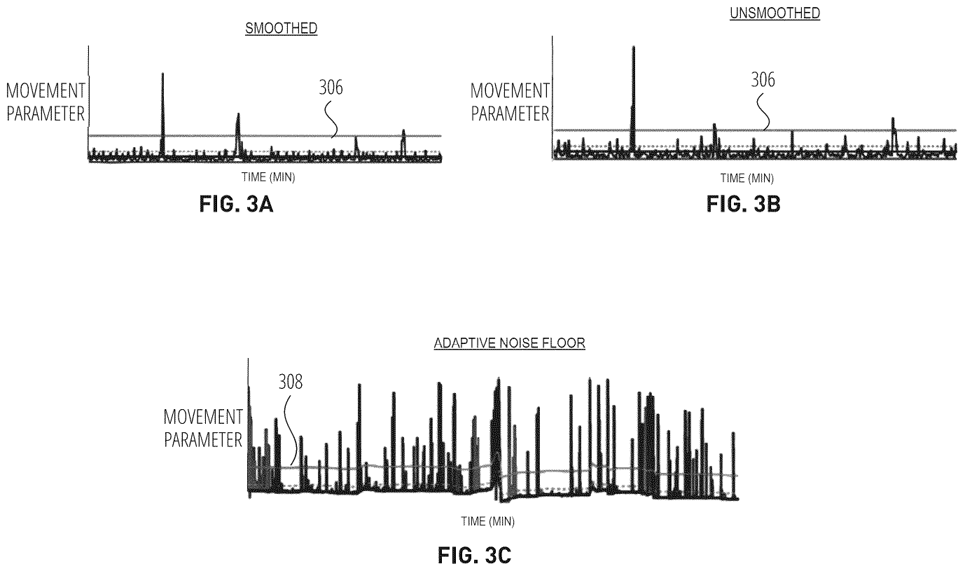

In an example, the processor circuitry 116 (as depicted in FIG. 1) can perform or otherwise control signal processing 220 to facilitate processing of received motion data 228, such as raw data from the IMU 114. For example, the signal processing 220 can facilitate filtering/preprocessing 222, e.g., via one or more filters (e.g., high-pass, low-pass, bandpass, notch, etc.) such as to reduce noise. In an example, the filtering/preprocessing 222 can apply a bandpass filter (e.g., a 5th order Butterworth bandpass filter) within a passband range of about 0.3 Hz and about 20 Hz. Herein, the term “about” constitutes a margin of +/−5% around recited values. In an example, the filtering/preprocessing 222 can facilitate summing respective vectors of a plurality of sensor axes, from the motion data, such as to calculate a vector magnitude identifying at least one candidate limb movement, based on the vector magnitude. FIG. 3A and FIG. 3B are charts showing smoothed motion data and unsmooth motion data, respectively, including a change in movement parameter (e.g., millivolts (u V) for EMG, gravitational constant (g) for accelerometer, degrees per second (deg/s) for gyroscope, etc.) over time. The filtering/preprocessing 222 can also facilitate smoothing the motion data 228 such as to further remove noise. For example, the smoothing can be optimized or otherwise augmented based on a training data set and can involve an about 0.1 second centered RMS envelope, selected such as to smooth the motion data 228 (e.g., the EMG signal or the IMU data) without removing potentially important signal information or significantly decreasing the signal power. FIG. 3C is a chart showing a change in movement parameter over time, including an adaptive noise floor. In an example, the filtering/preprocessing 222 can define an adaptive noise floor, adapted according to a specified, moving window during the therapy period. For example, the adaptive noise floor can include or use a moving window to calculate the Nth percentile (e.g., about a 70th percentile, about an 80th percentile, or about a 90th percentile) of the IMU signal during periods of rest (e.g., where the lower limbs of a patient are not moving).

Returning to FIG. 2B, the signal processing 220 can also facilitate classifying 224 of motion data 228 to classify a period of time as at least one of “potential leg movement” or “potential increased RLS symptoms”. Further, the classifying 224 can be performed by the processing circuitry 116 such as to define the start and end of candidate movements. In an example, the processing circuitry 116 can determining respective indications of corresponding individual limb movements based on a movement parameter from the motion data meeting one or more specified criteria, e.g., exceeding a specified (e.g., amplitude) threshold. For example, the classifying 224 can be performed such as to help optimize the amplitude threshold parameters, or additional amplitude criteria, such as based on cross-validation on a training data set.

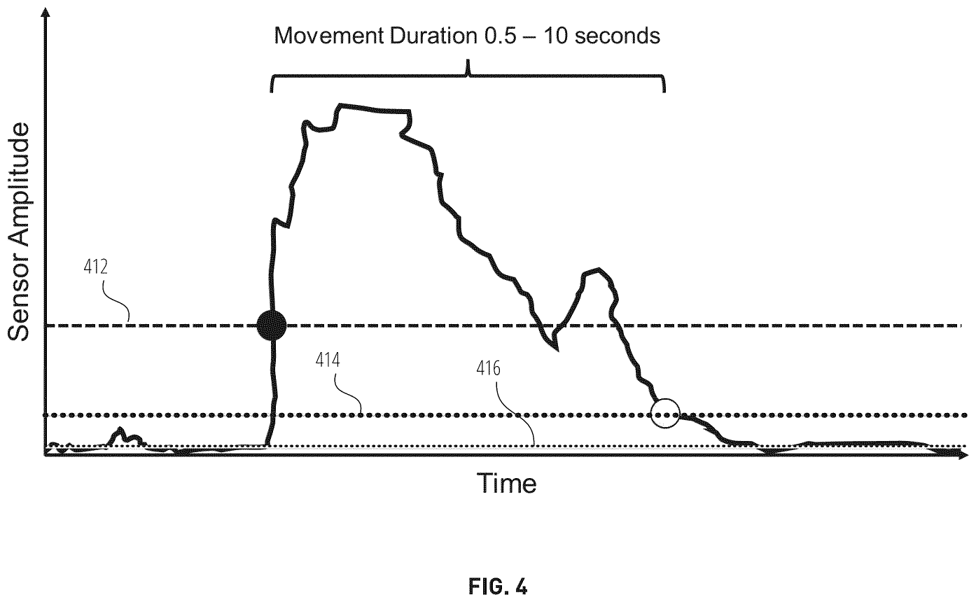

FIG. 4 is a chart showing a threshold-based technique for determining an indication of an individual limb movement. In an example, starting (e.g., starting movement threshold 412) and ending thresholds (e.g., ending movement threshold 414, the amplitude of the IMU signal above a noise floor or above the adaptive noise floor) can be optimized or otherwise augmented using an exhaustive grid search based on leave-one-out cross-validation on a training data set employing registered PSG technician (RPSGT) scoring of leg movements as the ground truth. For example, the starting threshold can be established as motion data exceeding a specified accelerometer threshold value, such as the specified accelerometer threshold value being within a range of about 0.01 gravitational force (g)-about 0.20 g and, and alternatively or additionally (such as concurrently) exceeding a specified gyroscope threshold value, the specified gyroscope threshold value being within a range of about 2 degrees per second (deg/s) and about 30 deg/s. Similarly, the ending threshold can be established as motion data falling beneath a specified accelerometer threshold value, the specified accelerometer threshold value being within a range of about 0.001 gravitational force (g)-about 0.1 g and, alternatively or additionally (such as concurrently) falling beneath a specified gyroscope threshold value, the specified gyroscope threshold value being within a range of 0.01 degrees per second (deg/s) and 15 deg/s. FIG. 4 depicts a hysteretic example in which the starting movement threshold value exceeds the ending movement threshold value by a hysteresis amount.

In an example, the classifying 224 can include employing fixed timing parameters such as to determine if a candidate movement is a “leg movement” and if a “leg movement” is a periodic leg movement (PLM). Candidate movements that are likely to be artifacts (such as based on computational criteria established on a training data set) can be removed during the classifying 224 to further improve specificity. A “rest period” can be defined herein as a period of the night in which the participant is resting (not moving). The rest period can be monitored and used to define a relative rest 416 parameter, establishing an expected sensor amplitude indicating that the patient is resting, and limb movements are not present. In an example, a rolling standard deviation (“rolling-SD”) can be calculated from the IMU in 10-second windows throughout the night to more clearly identify periods of activity (higher SD) that likely represent movement (i.e. not resting). The rolling minimum in 10 second centered windows can also be calculated from the motion data 228 such as to indicate where any substantial shifts in the resting IMU occurred (“shifts”). The “SD-threshold” can be specified as the ˜60th percentile of the rolling SD in each 30-second window and e.g., can be recalculated when a shift occurred. For example, periods where “rolling-SD” was less than “SD-threshold” can be considered “rest periods”.

In an alternative or additional exhaustive grid search, the number of consecutive samples can be optimized or otherwise augmented (or percentage of samples in a given time period) that needed to be above or below the noise floor that are needed to start or end, respectively, a movement. For example, the starting criteria and ending criteria can also be included in the grid search using range values of [1, 3, 5, 8, 10, 15, 20, 25] and percentages of [50%, 60%, 70%, 80%, 90%, 100%]. In example, such a grid search can be a 2-dimensional search or a 4-dimensional across a parameter space. In an example, the grid search can be a 4-dimensional search across the parameter space such as to optimize 2 sets of parameters. For example, a first set of parameters can include a starting threshold to a candidate movement. The first set of parameters can also include a number of samples specified to be above the starting threshold throughout the duration of the candidate movement. In an example, a second set of parameters can include an ending threshold to a candidate movement. The second set of parameters can also include a percentage of samples specified to be under the ending threshold in a specified period (e.g., about 0.5 seconds) after the proposed end of the movement for the movement to be considered complete.

In an example, the classifying 224 can involve testing various combinations of the 4 parameters such as to detect leg movements. For example, the classifying 224 can classify a start of a candidate leg movement as soon as one sample exceeds the starting. The classifying 224 can classify an end of the candidate leg movement as soon as percent under end percentage of leg movements in the specified period (e.g., the coming 0.5 seconds) of the signal are under the ending threshold. For example, if the classifying 224 involves sampling at about 52 Hz and have a percent under end value of about 50%, the classifying can specify that at least 13 out of the 26 samples in the next ˜0.5 seconds must be under the ending movement threshold to determine an end of the candidate leg movement. Once the start and end of the candidate leg movement are determined, the classifying 224 can ensure at least a “samples over start” number of samples during the leg movement are greater than the starting threshold. For example, it there are fewer than “samples over start” samples over the starting movement threshold, then the classifying 224 can involve discarding the candidate leg movement.

In an example, the ranges for the parameters of the first and second set can be specified according to table 1:

TABLE 1

| Starting Threshold | Ending Threshold | Samples Over Start | Percent Under End |
|---|---|---|---|
| Each of | Each of | Within a range of | Within a range of |
| (concurrently): | (concurrently): | about 1 to about 25 | about 50% to about |
| Accelerometer: | Accelerometer: | samples | 100% |
| Within a range of | Within a range of | | |
| about 0.1 g to about | about 0.01 g to | | |
| 0.20 g | about 0.1 g | | |
| Gyroscope: Within | Gyroscope: within | | |
| a range of about 2 | a range of about 1 | | |
| deg/sec to about 30 | deg/sec to about 15 | | |
| deg/sec | deg/sec | | |

In an example, to help optimize or select the grid search parameters, a leave-one-out cross-validation on the training data set (with RPSGT scoring as ground truth) can be performed. Such desired parameters can be selected optimizing or augmented for one or more of Accuracy, Sensitivity, combined Sensitivity-Accuracy, or F1-score. Such an approach can facilitate that all movements are detected (high sensitivity), while minimizing, e.g., false positive movements (high accuracy and precision). Cross-validation can also be employed such as to help avoid overfitting.

In an example, the classifying 224 can be performed using a low-complexity (minimal processing capability and low power consumption) algorithm. For example, such low-complexity classification can be based on one or more threshold crossings or based on signal amplitude. For example, the one or more threshold crossings can include threshold crossings from a leg movement signal (e.g., a rectified or preprocessed signal) from a motion sensor. In an example, the filtered sensor data classifying a period of time as at least one of “potential leg movement” or “potential increased RLS symptoms” can define a first subset of motion data 228.

In an example, the signal processing 220 can facilitate feature selection 226, e.g., of the first subset of motion data 228. The feature selection 226 can calculate and save a feature set. This can help reduce memory usage as compared to storing raw data and can also provide an input to train a machine learning model. The feature selection 226 can calculate a feature set for each IMU axes, vector magnitude, or a composite of each axis. In an example, the feature set can include one or more statistics that describe the signal amplitude or other characteristics, such as mean, root mean square, percentiles, maximum, minimum, range, maximum to mean, area under the curve, variation, kurtosis, skewness, or correlation coefficients between signal values corresponding to different IMU axes. The feature set can also include a description of starting, ending, maximum, minimum, changes in position, and rate of changes in position of the IMU 114 using the gravitational vector and/or angle of inclination.

In an example, the feature selection 226 can involving transforming raw IMU data into features, resulting in data compression (e.g., between about 10× and about 1000×). Such a transformation can be implemented, e.g., directly in embedded software on the PCBA of the wearable electrostimulation device 102 (as depicted in FIG. 1) and can involve relatively low or manageable data storage and data transfer requirements.

In an example, the feature selection 226 can include estimating occurrences of PLMs based on the features. This can be implemented on the embedded software of the wearable electrostimulation device 102 or, e.g., on a separate computing platform with even greater computational capabilities than available on the PCBA (e.g. on a cloud server or other connected device) following data transfer from the PCBA to such a system. For example, the feature selection 226 can include identifying a plurality of candidate limb movements and comparing the plurality of candidate limb movements with a reference data set that includes candidate limb movements that have been validated as periodic limb movements (PLMs), such as to generate an indication of whether an individual candidate limb movement is likely to represent a PLM as distinguished from an artifact.

In an example, the feature selection 226 can include a random forest machine learning model (“IMU-ML” algorithm), which can be trained using features extracted from 2-second windows for the pre-processed data from each IMU sensor (accelerometer, gyroscope). Additionally, the gravitational vector of the signal can be extracted, such as using a ˜0.25 Hz lowpass filter (e.g., a 0.25 Hz 2^{nd} order Butterworth lowpass filter). Features can include signal statistics (e.g., maximum, root mean square), frequency domain information (e.g., maximum spectral power, power and frequency ratios), positions (e.g., gravitational vector value, angles of inclination), and others (e.g., wavelet decomposition, threshold crossings, cross-correlations). For machine learning model training and feature selection, features for each 2-second window can be aggregated into 60-minute windows to predict ground truth PLM index (PLMs per hour) for each hour in the data set. Nested, leave-one-out cross-validation can used for feature selection and model evaluation so that the IMU-ML model results can be unbiased and generalizable to the larger population.

In an example, the feature set can also include one or more features derived from the frequency domain of the signal, e.g., obtained using a fast Fourier transform, such as dominant frequency, power at the dominant frequency, total power, ratio of power at the dominant frequency to total power, frequency bandwidth, spectral centroid, and mean or median frequency. Such features can be derived from only the low frequency range where human movement is most likely to occur or alternatively across all available frequencies. The feature set can also include median/zero crossings, Lempel-Ziv Complexity, entropy rate, and the maximum Lyapunov Exponent, or other features describing the time-frequency domain, e.g., wavelet decompositions and wavelet entropy.

In an example, the calculated feature set can be as an input to train a machine learning algorithm, e.g., to predict a range of target HF electrostimulation signal parameters for delivery to the patient that will be subthreshold to the patient waking while mitigating the RLS or PLMD symptom. The machine learning algorithm can be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method.

In an example, a regression model is used, and the model is a vector of coefficients corresponding to a learned importance for each of the features of the feature set. In an example, the machine learning algorithm can implement a regression problem (e.g., linear, polynomial, regression trees, kernel density estimation, support vector regression, random forests implementations, or the like).

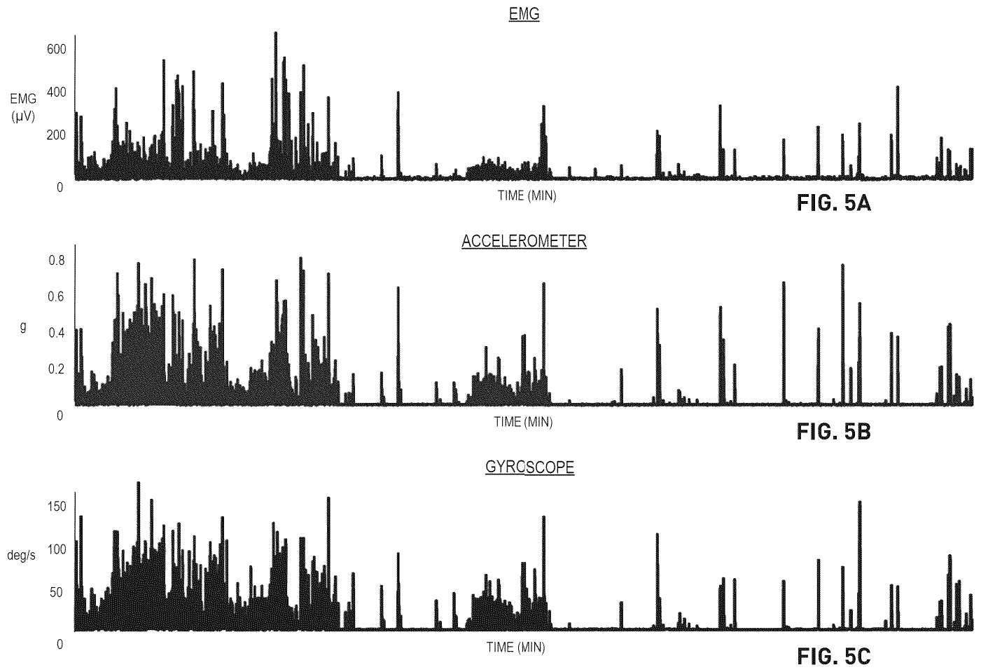

FIG. 5A, FIG. 5B, and FIG. 5C are charts showing electromyography (EMG), accelerometer, and gyroscope movement parameters plotted over time, respectively. In one approach to receiving feedback from electrostimulation delivered to the patient, the RLS treatment system can include at least one EMG electrode for detecting electrical activity in a muscle of a patient limb. IMU data (e.g., gyroscope velocity over time or accelerometer g) can resemble rectified voltage data from an EMG electrode. As such, IMU data can be used to approximate electrical activity in a muscle of a patient limb without necessarily needing to receive EMG data as a part of the motion data 228 (as described with respect to FIG. 2B). Such approximation can provide advantages over certain EMG approaches, such as with regard to power saving and patient ease of obtaining the measurements in an in-home treatment setting.

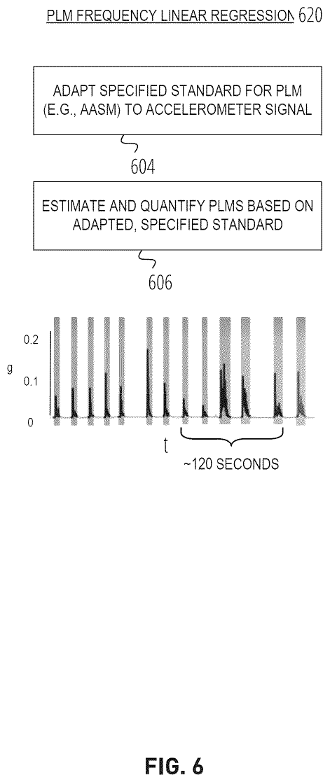

FIG. 6 is a chart showing several processes to estimating a patient symptom of Restless Legs Syndrome during concurrent delivery of an electrostimulation waveform to the patient. Any of the respective steps of process 618, process 620, and process 622 can be performed on the processor circuitry 116, processor 902, or other componentry described herein.

In an example, a logarithmic motion linear regression process 618 can be performed. For example, at 602, the process 618 can include calculating a logarithm (log) of a mean or other central tendency of an accelerometer vector magnitude over a specified period of time (e.g., over about 100 seconds(s), about 110 s, about 120 s, about 130 s, etc. In an example, the motion data can include accelerometer data without a need for receiving any gyroscopic or EMG data.

In an example, a periodic limb movement (PLM) frequency linear regression process 620 can be performed. For example, at 604, the process 620 can include adapting a specified standard for classifying a PLM to the accelerometer data. For example, the specified standard can be according to limb movement criteria (e.g., a timing, frequency, etc. of a qualifying candidate limb movement) established the American Academy of Sleep Medicine (AASM), or alternatively or additionally specified according to limb movement criteria established by the World Association of Sleep Medicine (WASM). At 606, the process can include estimating and quantifying a plurality of PLMs based on the adapted, specified standard.

In an example, a feature-based machine learning process 622 can be performed. At 608 and 610, the process 622 can include additional feature-based machine learning operations. At 608, the process can include extracting, via a trained machine learning model, a plurality of IMU signal features from a first specified period (e.g., about 1 second, about 2 seconds, about 3 seconds, about 5 seconds, etc.). Features can include signal statistics (e.g., maximum, root mean square (rms)), frequency domain information (e.g., maximum spectral power, power and frequency ratios), positions (e.g., gravitational vector value, angles of inclination), and others (e.g., wavelet decomposition, threshold crossings, cross-correlations).

At 610, the process 600 can include aggregating the features extracted in operation 608 over a second specified period (e.g., about 2 minutes (min), about 3 min, about 4 min, about 5 min, about 10 min, about 15 min, etc.

In an example, the PLM frequency linear regression 620 and a feature-based machine learning process can be performed together (e.g., successively), such that an output of the PLM frequency linear regression 620 provides an input to the feature-based machine learning process. For example, process 620 can include quantifying a number of PLMs within a time window and use the resulting quantity as one of the features for the trained ML model of process 622. Also process 620 can include detecting a plurality of PLMs and calculate a plurality of features for each of the plurality of PLMs, and the resulting features can be used as inputs to the trained ML model of process 622. Here, the trained ML model can also receive as an input at least one feature related to a non-periodic leg movement, such as a voluntary leg movement. In an example, at least one of statistical, time-domain, or frequency-domain features of an individual limb movement can be identified via the linear regression 620. Here, a machine learning (ML) model can be trained, using the on the statistical, time-domain, or frequency-domain features as training data, to identify limb movements in motion data from an IMU.

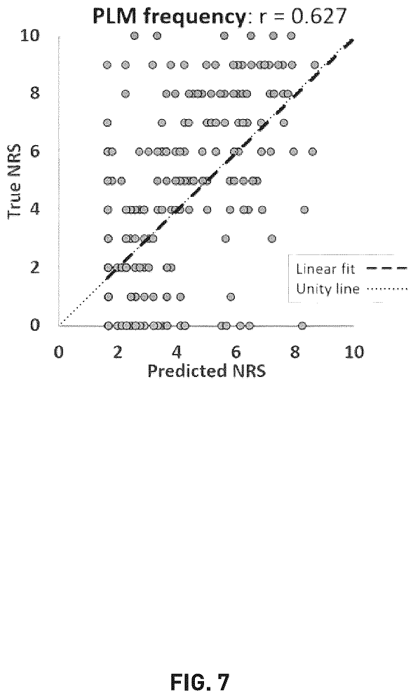

FIG. 7 is a chart showing a PLM frequency linear regression process (e.g., process 620) for predicting RLS severity (NRS ratings), as compared with ground truth data. In an example, process 620 demonstrates a correlation between estimated NRS and patient reported NRS. The data set for IMU and RLS severity NRS ratings can be collected, e.g., from patients with moderate-to-severe RLS during a suggested immobilization test (SIT) procedure.

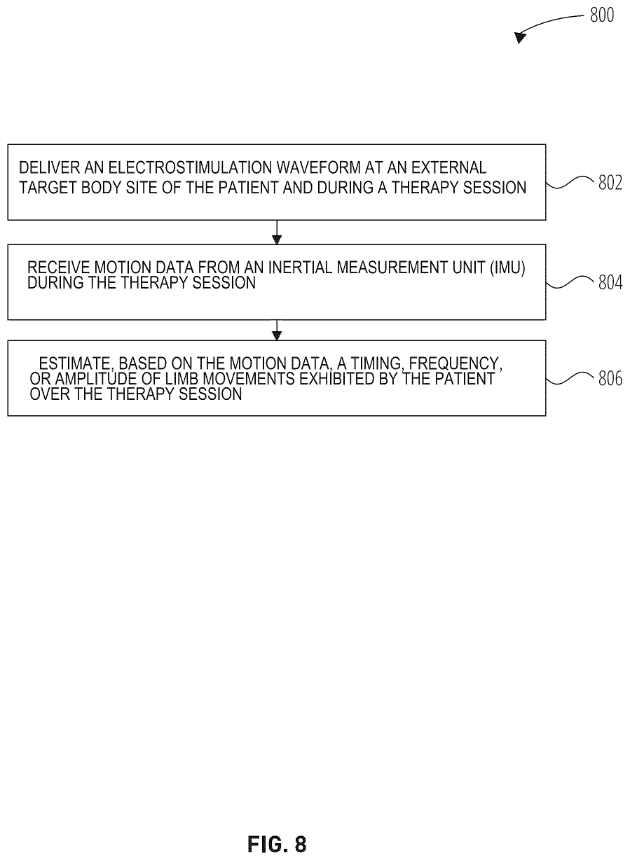

FIG. 8 is a flowchart describing a process for detecting patient limb movement during a transcutaneous therapy session that includes delivery of neurostimulation therapy on a human patient.

The process 800 can include, at step 802, delivering an electrostimulation waveform to an external target body site of a patient during a therapy session. This electrostimulation waveform can be generated via an electrostimulation electronics unit as having specified signal characteristics. For example, the waveform can be controlled such as to have a frequency within a range of 500 hertz (Hz) to 10,000 kilohertz (kHz), an average root-mean squared current greater than 5 milliamps (mAᵣₘₛ) (e.g., greater than 10 mAᵣₘₛ), or both.

At step 804, the process 800 can include receiving motion data from an inertial measurement unit (IMU) during the therapy session that includes the delivery of the electrostimulation waveform. For example, the IMU can capture detailed information about the patient's limb movements throughout the therapy session. In an example, the receiving motion data can be concurrent with the delivery of electrostimulation to the external target body site.

At step 806, the process 800 can include estimating, based on the motion data, a timing, frequency, or amplitude of limb movements exhibited by the patient over the therapy session. This estimation process involves analyzing the collected motion data to identify specific movement patterns and characteristics. In an example, the estimating can involve an algorithm to identify certain types of movements, such as periodic limb movements (PLMs) or voluntary movements made by the patient to relieve RLS symptoms. In an example, the process 800 can include establishing or adjusting at least one parameter of the electrostimulation waveform based on this estimation, allowing for dynamic, personalized treatment protocols. Specifically in examples including identifying PLMs, the process 800 can adhere to specified PLM standards, ensuring consistency and reliability in movement classification. Additionally, the system can distinguish between PLMs and voluntary movements made by the patient to alleviate RLS symptoms, providing valuable insights into the patient's condition and treatment efficacy.

In an example, to enhance the accuracy of movement detection, the process 800 can include a thresholding step. For example, individual limb movements can be identified when movement parameters from the motion data exceed specified thresholds. The thresholding step can specify different starting and ending thresholds for more precise movement detection. For example, a starting threshold can be established such that motion data exceeding both a specified accelerometer threshold (within a range of 0.01 g to 0.20 g) and a specified gyroscope threshold (within a range of 2 deg/s to 30 deg/s). Similarly, the ending threshold can be established such that it is traversed when the motion data falls beneath specified accelerometer (0.001 g to 0.1 g) and gyroscope (0.01 deg/s to 15 deg/s) values.

In an example, the process 800 can include further data processing steps to improve movement detection accuracy. Such steps (e.g., filtering or smoothing the motion data) can help reduce noise and enhance signal quality. Additionally, the process can include determining a vector magnitude corresponding with the IMU data, such as summing respective vectors from multiple sensor axes, such as to provide a comprehensive measure of movement intensity. This vector magnitude can be used such as to identify candidate limb movements.

In an example, the process 800 can include comparing certain identified candidate limb movements with a reference data set of validated PLMs. This comparison can help distinguish a genuine PLMs from artifacts or other non-relevant movements, increasing the overall accuracy of the process 800. The process 800 can also involve adaptive techniques to account for varying conditions during the therapy session. For instance, the process 800 can include establishing an adaptive noise floor, e.g., that adjusts according to a specified moving window throughout the therapy period.

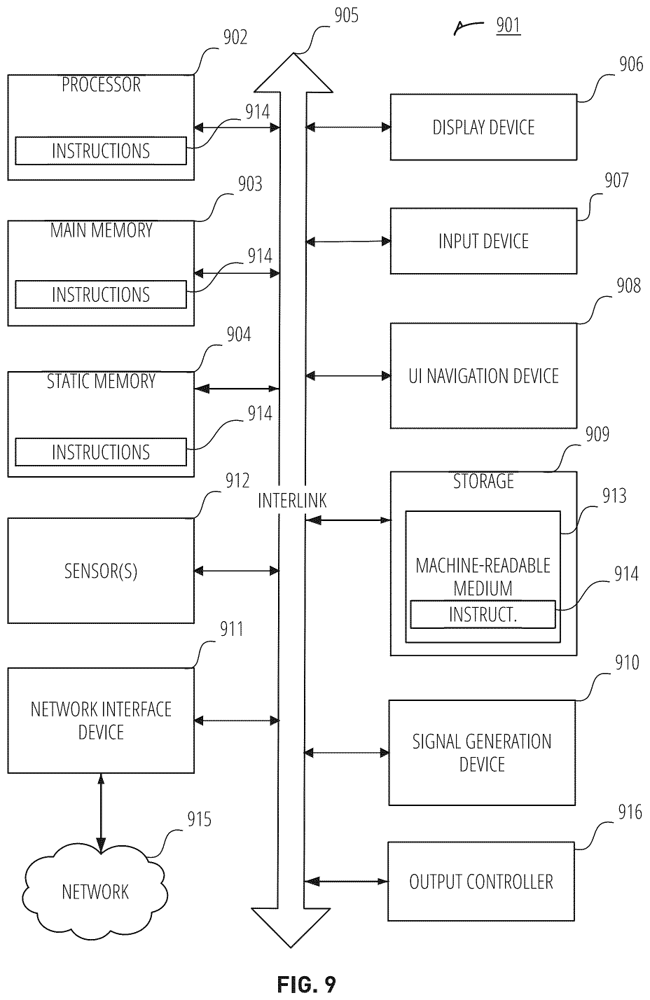

FIG. 9 illustrates generally an example of a block diagram of a machine 901 upon which any one or more of the techniques (e.g., methodologies) discussed herein may perform in accordance with some examples. In alternative embodiments, the machine 901 may operate as a standalone device or may be connected (e.g., networked) to other machines. In a networked deployment, the machine 901 may operate in the capacity of a server machine, a client machine, or both in server-client network environments. In an example, the machine 901 may act as a peer machine in peer-to-peer (P2P) (or other distributed) network environment. The machine 901 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the executions units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) 901 may include a hardware processor 902 (e.g., a central processing unit (CPU), a graphics processing unit (GPU), a hardware processor core, or any combination thereof), a main memory 903 and a static memory 904, some or all of which may communicate with each other via an interlink (e.g., bus) 905. The machine 901 may further include a display unit 906, an alphanumeric input device 907 (e.g., a keyboard), and a user interface (UI) navigation device 908 (e.g., a mouse). In an example, the display unit 906, alphanumeric input device 907 and UI navigation device 908 may be a touch screen display. The machine 901 may additionally include a storage device (e.g., drive unit) 909, a signal generation device 910 (e.g., a speaker), a network interface device 911, and one or more sensors 912, such as a global positioning system (GPS) sensor, compass, accelerometer, or other sensor. The machine 901 may include an output controller 916, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 909 may include a machine readable medium 913 that is non-transitory on which is stored one or more sets of data structures or instructions 914 (e.g., software) embodying or utilized by any one or more of the techniques or functions described herein. The instructions 914 may also reside, completely or at least partially, within the main memory 903, within static memory 904, or within the hardware processor 902 during execution thereof by the machine 901. In an example, one or any combination of the hardware processor 902, the main memory 903, the static memory 904, or the storage device 909 may constitute machine readable media.

While the machine readable medium 913 is illustrated as a single medium, the term “machine readable medium” may include a single medium or multiple media (e.g., a centralized or distributed database, or associated caches and servers) configured to store the one or more instructions 914.

The term “machine readable medium” may include any medium that is capable of storing, encoding, or carrying instructions for execution by the machine 901 and that cause the machine 901 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 914 may further be transmitted or received over a communications network 915 using a transmission medium via the network interface device 911 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 911 may include one or more physical jacks (e.g., Ethernet, coaxial, or phone jacks) or one or more antennas to connect to the communications network 915. In an example, the network interface device 911 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 901, and includes digital or analog communications signals or other intangible medium to facilitate communication of such software.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method for detecting patient limb movement during a transcutaneous therapy session that includes delivery of neurostimulation therapy on a human patient, the method comprising:
delivering an electrostimulation waveform, generated via an electrostimulation electronics unit with a frequency within a range of 500 hertz (Hz) to 10,000 kilohertz (kHz) with an average root-mean squared current greater than 5 milliAmps (mAᵣₘₛ), at an external target body site of the patient and during a therapy session;
receiving motion data from an inertial measurement unit (IMU) during the therapy session that includes the delivery of the electrostimulation waveform; and
estimating, based on the motion data, at least one of a timing, frequency, or amplitude of limb movements exhibited by the patient over the therapy session, wherein the estimation of the timing, frequency, or amplitude of limb movements includes determining respective indications of corresponding individual limb movements based on a movement parameter from the motion data exceeding a specified threshold.

2. The method of claim 1, comprising establishing or adjusting at least one parameter of the electrostimulation waveform based on the estimation of at least one of the timing, frequency, or amplitude of limb movements.

3. The method of claim 1, wherein the estimating the timing, frequency or amplitude of limb movements includes identifying at least one limb movement as a periodic limb movement (PLM), indicating movement parameters according to a specified PLM standard.

4. The method of claim 1, wherein the estimating the timing, frequency, or amplitude of limb movements includes identifying at least one limb movement as indicative of a voluntary movement made by the patient to relieve a symptom of restless legs syndrome (RLS).

5. The method of claim 1, wherein the estimating the timing, frequency, or amplitude of limb movements includes identifying at least one limb movement as indicative of a voluntary movement made by the patient that indicates a state of arousal or awakening.

6. The method of claim 1, comprising estimating at least one symptom of restless legs syndrome (RLS) or periodic limb movement disorder (PLMD) based on the estimated timing and frequency of limb movements exhibited by the patient over the therapy session.

7. The method of claim 1, wherein the specified threshold includes a starting threshold and ending threshold, different from the starting threshold.

8. The method of claim 1, comprising:
identifying at least one of statistical, time-domain, or frequency-domain features of an individual limb movement; and
training a machine learning (ML) model, using the on the statistical, time-domain, or frequency-domain features as training data, to identify limb movements in motion data from an IMU.

9. The method of claim 7, wherein the starting threshold is established as motion data exceeding a specified accelerometer threshold value, the specified accelerometer threshold value being within a range of 0.01 gravitational force (g)-0.20 g and, concurrently exceeding a specified gyroscope threshold value, the specified gyroscope threshold value being within a range of 2 degrees per second (deg/s) and 30 deg/s.

10. The method of claim 7, wherein the ending threshold is established as motion data falling beneath a specified accelerometer threshold value, the specified accelerometer threshold value being within a range of 0.001 gravitational force (g)-0.1 g and, concurrently falling beneath a specified gyroscope threshold value, the specified gyroscope threshold value being within a range of 0.01 degrees per second (deg/s) and 15 deg/s.

11. The method of claim 1, comprising:
at least one of filtering or smoothing the motion data;
summing respective vectors of a plurality of sensor axes, from the motion data, to calculate a vector magnitude; and
identifying at least one candidate limb movement, based on the vector magnitude.

12. The method of claim 11, comprising:
identifying a plurality of candidate limb movements; and
comparing the plurality of candidate limb movements with a reference data set that includes candidate limb movements that have been validated as periodic limb movements (PLMs), to generate an indication of whether an individual candidate limb movement is likely to represent a PLM as distinguished from an artifact.

13. The method of claim 1, wherein the specified threshold includes a specified number of samples or a specified percentage of samples detected above or beneath a noise floor, to determine an indication of an individual limb movement.

14. The method of claim 13, wherein the noise floor is an adaptive noise floor, adapted according to a specified, moving window during the therapy session.

15. A non-transitory computer-readable storage medium, the computer-readable storage medium including instructions that when executed by a computer, cause the computer to:
deliver an electrostimulation waveform, generated via an electrostimulation electronics unit with a frequency within a range of 500 hertz (Hz) to 10,000 kilohertz (kHz) with an average root-mean squared current greater than 5 milliAmps (mAᵣₘₛ), at an external target body site of a patient and during a therapy session;
receive motion data from an inertial measurement unit (IMU) during the therapy session that includes the delivery of the electrostimulation waveform; and
estimate, based on the motion data, at least one of a timing, frequency, or amplitude of limb movements exhibited by the patient over the therapy session, wherein the estimation of the timing, frequency, or amplitude of limb movements includes determining respective indications of corresponding individual limb movements based on a movement parameter from the motion data exceeding a specified threshold.

16. The computer-readable storage medium of claim 15, wherein the estimating the timing, frequency, or amplitude of limb movements includes identify at least one limb movement as a periodic limb movement (PLM), indicating movement parameters according to a specified PLM standard.

17. The computer-readable storage medium of claim 15, wherein the estimating the timing, frequency, or amplitude of limb movements includes identify at least one limb movement as indicative of a voluntary movement made by the patient to relieve a symptom of restless legs syndrome (RLS).

18. The computer-readable storage medium of claim 15, wherein the computer-readable storage medium includes instructions that when executed by a computer, cause the computer to estimate at least one symptom of restless legs syndrome (RLS) or periodic limb movement disorder (PLMD) based on the estimated timing and frequency of limb movements exhibited by the patient over the therapy session.

19. The computer-readable storage medium of claim 15, wherein the computer-readable storage medium includes instructions that when executed by a computer, cause the computer to:
at least one of filter or smooth the motion data;
sum respective vectors of a plurality of sensor axes, from the motion data, to calculate a vector magnitude; and
identify at least one candidate limb movement, based on the vector magnitude.

20. The computer-readable storage medium of claim 19, wherein the computer-readable storage medium includes instructions that when executed by a computer, cause the computer to:
identify a plurality of candidate limb movements; and
compare the plurality of candidate limb movements with a reference data set that includes candidate limb movements that have been validated as periodic limb movements (PLMs), to generate an indication of whether an individual candidate limb movement is likely to represent a PLM as distinguished from an artifact.

21. An apparatus for detecting patient limb movement during a transcutaneous therapy session that includes delivery of neurostimulation therapy on a human patient, the apparatus comprising:
means for delivering an electrostimulation waveform, generated via an electrostimulation electronics unit with a frequency within a range of 500 hertz (Hz) to 10,000 kilohertz (kHz) with an average root-mean squared current greater than 5 milliAmps (mAᵣₘₛ), at an external target body site of the patient and during a therapy session;
means for receiving motion data from an inertial measurement unit (IMU) during the therapy session that includes the delivery of the electrostimulation waveform; and
means for estimating, based on the motion data, at least one of a timing, frequency, or amplitude of limb movements exhibited by the patient over the therapy session, wherein the estimation of the timing, frequency, or amplitude of limb movements includes determining respective indications of corresponding individual limb movements based on a movement parameter from the motion data exceeding a specified threshold.

22. The apparatus of claim 21, comprising means for establishing or adjusting at least one parameter of the electrostimulation waveform based on the estimation of at least one of the timing, frequency, or amplitude of limb movements.
